(19) United States
(12) Patent Application Publication
(10) Pub. No.: US 2024/0316332 A1
(43) Pub. Date: Sep. 26, 2024

(54) Mechanical Cardiac Support Device And Methods Of Using Same

(71) Applicant: United States Government As Represented By The Department Of Veterans Affairs, Washington, DC (US)

(72) Inventor: Kartik Mani, St. Louis, MO (US)

(21) Appl. No.: 18/613,582

(22) Filed: Mar. 22, 2024

Related U.S. Application Data
(60) Provisional application No. 63/491,585, filed on Mar. 22, 2023.

(51) Int. Cl. A61M 60/13; A61M 60/165; A61M 60/237; A61M 60/414; A61M 60/508; A61M 60/861; A61M 60/867; A61M 60/873

(52) U.S. Cl. CPC A61M 60/13; A61M 60/165; A61M 60/237; A61M 60/414; A61M 60/508; A61M 60/861; A61M 60/867; A61M 60/873; A61M 2205/3365

(57) ABSTRACT

A portion of a catheter is positionable within a heart having a left atrium, an aorta, and a septum. The catheter includes an elongate body having a first portion that is configured to be positioned within a left atrium and a second portion that is distal of the first portion and configured to be positioned in the aorta when the first portion is positioned within the left atrium. Each of the first portion and the second portion defining at least one opening extending from an outer surface to an inner surface of the elongate body. An impeller is configured to effect blood flow through the elongate body from the first portion to the second portion. First, second, and third anchors secure the catheter in position within the heart.

### CROSS-REFERENCE TO RELATED APPLICATION

This application claims priority to and the benefit of the filing date of U.S. Provisional Patent Application No. 63/491,585, filed Mar. 22, 2023, the entirety of which is hereby incorporated by reference herein.

### FIELD

This disclosure relates to devices for providing cardiac support.

### BACKGROUND

Improvements in technology have resulted in improved acute salvage of cardiac diseases. Similarly, advancements in long term mechanical cardiac support (MCS) have somewhat offset the critical lack of donors for orthotopic cardiac transplant (OCT) by providing options to New York Heart Association (NYHA) Class IV patients with severely decreased left ventricle function, refractory to currently available medical and device support measures. However, a significant number of MCS candidates are not felt to be candidates for MCS implantation due to frailty, comorbidities with multi-organ dysfunction, social issues, prior thoracic surgery or disease. While some of these patients are better served with palliative management, many of these patients could benefit from a “bridge-to-reconditioning” for possibly future definitive MCS or OCT.

### SUMMARY

Described herein, in various aspects, is a catheter comprising a portion that is positionable within a heart having a left atrium, an aorta, and a septum. The catheter comprises an elongate body having an outer surface and an inner surface defining an interior passage of the elongate body. The elongate body comprises a first portion that is configured to be positioned within a left atrium, the first portion defining at least one opening extending from the outer surface to the inner surface of the elongate body. The elongate body further comprises a second portion that is distal of the first portion and configured to be positioned in the aorta when the first portion is positioned within the left atrium, the second portion defining at least one opening extending from the outer surface to the inner surface of the elongate body. An impeller is positioned between the first portion and the second portion. The impeller is configured to effect blood flow through the elongate body from the first portion to the second portion. A first anchor is positioned proximal of the second portion. The first anchor is configured to inhibit movement of the second portion proximally past the aortic valve. A second anchor is positioned proximal of the first portion. The second anchor is configured to inhibit movement of the first portion of the elongate body proximally past the septum. A third anchor positioned proximally of the second anchor. The third anchor is configured to inhibit movement of the first portion of the elongate body distally.

Methods of implanting the catheter are also disclosed.

Additional advantages of the invention will be set forth in part in the description that follows, and in part will be obvious from the description, or may be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

### DESCRIPTION OF THE DRAWINGS

These and other features of the preferred embodiments of the invention will become more apparent in the detailed description in which reference is made to the appended drawings wherein:

FIG. 1 is a schematic diagram of a catheter, as disclosed herein, implanted in a heart.

FIG. 2 is a schematic diagram of the catheter of FIG. 1

FIG. 3 is a schematic diagram of a system comprising the catheter of claim 1.

FIG. 4 is a schematic diagram of an exemplary of a kit for positioning the catheter.

### DETAILED DESCRIPTION

The present invention now will be described more fully hereinafter with reference to the accompanying drawings, in which some, but not all embodiments of the invention are shown. Indeed, this invention may be embodied in many different forms and should not be construed as limited to the embodiments set forth herein; rather, these embodiments are provided so that this disclosure will satisfy applicable legal requirements. Like numbers refer to like elements throughout. It is to be understood that this invention is not limited to the particular methodology and protocols described, as such may vary. It is also to be understood that the terminology used herein is for the purpose of describing particular embodiments only, and is not intended to limit the scope of the present invention.

Many modifications and other embodiments of the invention set forth herein will come to mind to one skilled in the art to which the invention pertains having the benefit of the teachings presented in the foregoing description and the associated drawings. Therefore, it is to be understood that the invention is not to be limited to the specific embodiments disclosed and that modifications and other embodiments are intended to be included within the scope of the appended claims. Although specific terms are employed herein, they are used in a generic and descriptive sense only and not for purposes of limitation.

As used herein the singular forms “a,” “an,” and “the” include plural referents unless the context clearly dictates otherwise. For example, use of the term “an opening” can refer to one or more of such openings, and so forth.

All technical and scientific terms used herein have the same meaning as commonly understood to one of ordinary skill in the art to which this invention belongs unless clearly indicated otherwise.

As used herein, the terms “optional” or “optionally” mean that the subsequently described event or circumstance may or may not occur, and that the description includes instances where said event or circumstance occurs and instances where it does not.

The word “or” as used herein means any one member of a particular list and, except where context dictates otherwise, can also include any combination of members of that list.

As used herein, the term “at least one of” is intended to be synonymous with “one or more of.” For example, “at least one of A, B and C” explicitly includes only A, only B, only C, and combinations of each.

Ranges can be expressed herein as from “about” one particular value, and/or to “about” another particular value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint. Optionally, in some aspects, when values are approximated by use of the antecedent “about,” it is contemplated that values within up to 15%, up to 10%, up to 5%, or up to 1% (above or below) of the particularly stated value can be included within the scope of those aspects. Similarly, if further aspects, when values are approximated by use of “approximately,” “substantially,” and “generally,” it is contemplated that values within up to 15%, up to 10%, up to 5%, or up to 1% (above or below) of the particularly stated value can be included within the scope of those aspects. In still further aspects, when angular relationships (e.g., “parallel” or “perpendicular”) are approximated by use of “approximately,” “substantially,” or “generally,” it is contemplated that angles within 15 degrees (above or below), within 10 degrees (above or below), within 5 degrees (above or below), or within 1 degree (above or below) of the stated angular relationship can be included within the scope of those aspects.

As used herein, “French” refers to a measurement of ⅓ of a millimeter.

As used herein, “proximal” refers to a portion of a device facing or closest to a clinician, and “distal” refers to a portion of the device facing or closest to the patient. The distal end portion of a catheter can, thus, be understood to be an insertional portion of the catheter that is the leading portion of the catheter when the catheter is advanced within the body of a patient as disclosed herein.

It is to be understood that unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is in no way intended that an order be inferred, in any respect. This holds for any possible non-express basis for interpretation, including: matters of logic with respect to arrangement of steps or operational flow; plain meaning derived from grammatical organization or punctuation; and the number or type of aspects described in the specification.

The following description supplies specific details in order to provide a thorough understanding. Nevertheless, the skilled artisan would understand that the apparatus, system, and associated methods of using the apparatus can be implemented and used without employing these specific details. Indeed, the apparatus, system, and associated methods can be placed into practice by modifying the illustrated apparatus, system, and associated methods and can be used in conjunction with any other apparatus and techniques conventionally used in the industry.

The devices described herein can be used for providing assistance to individuals suffering from severe cardiac disease. The disclosed catheter can comprise a percutaneously inserted transseptal device using a large caliber impeller to drive flow from the left atrium to the aorta. As shown in the drawings, utilization of venous insertion allows for use of large catheters and devices with a much lower risk of vascular issues as compared to conventional alternatives (e.g., a ventricular assist device), especially for long term device use, such as for several months or years at a time.

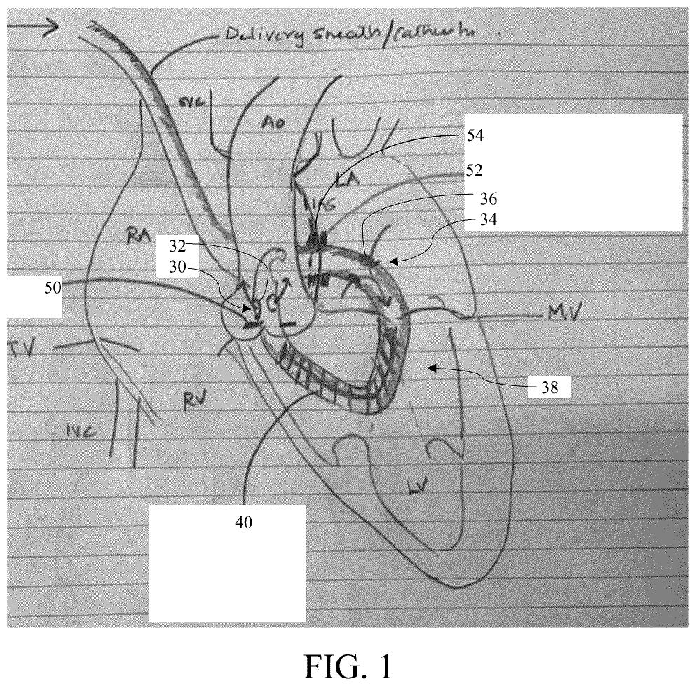

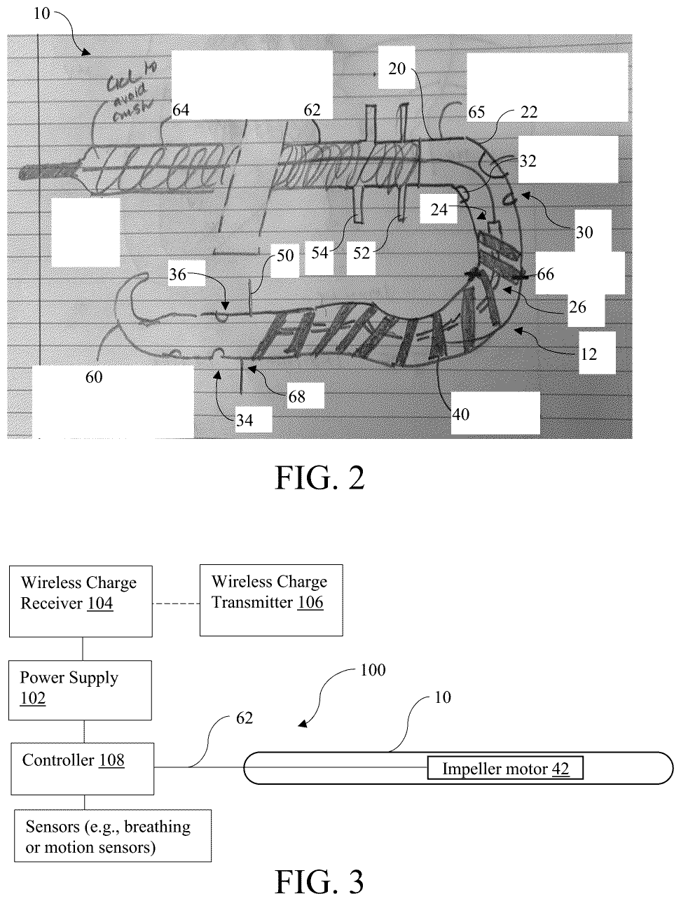

Disclosed herein, and with reference to FIGS. 1-2, is a catheter 10 comprising a portion 12 that is positionable within a heart having a left atrium (LA), an aorta (AO), and a septum (IAS). The catheter 10 can comprise an elongate body 20 having an outer surface 22 and an inner surface 24 defining an interior passage 26 of the elongate body.

The elongate body 20 (referred to herein also as “the body”) can comprise a first portion 30 that is configured to be positioned within a left atrium. The first portion 30 can define at least one opening 32 extending from the outer surface 22 to the inner surface of the body 24. In some aspects, the at least one opening 32 can comprise a plurality of openings 32. In some aspects, openings 32 of the plurality of openings 32 can be spaced axially and/or circumferentially from other openings of the plurality of openings 32. In some aspects, the first portion 30 can be defined as a length of the elongate body 20 across which the opening(s) 32 are positioned.

The elongate body 20 can further comprise a second portion 34 that is distal of the first portion 30 and configured to be positioned in the aorta when the first portion 34 is positioned within the left atrium. The second portion 34 can define at least one opening extending 36 from the outer surface 22 to the inner surface 24 of the body 20. In some aspects, the second portion 34 can be defined as a length of the elongate body 20 across which the opening(s) 36 are positioned.

An impeller 40 can be positioned within the interior passage 26 of the body 20 between the first portion 30 and the second portion 34. The impeller 40 is configured to effect blood flow through the elongate body 20 from the first portion 30 to the second portion 34. Accordingly, when the catheter 10 is positioned within the heart, the impeller 40 can be configured to effect blood flow from blood in the left atrium that is external to the outer surface 22 of the body, through the at least one opening 32, through the interior passage 26, out the at least one opening 36, and into the aorta. In exemplary aspects, the impeller can comprise a driven rotor that effects axial blood flow as described herein. In some aspects, the impeller can comprise a drive shaft that couples to the driven rotor. The drive shaft can be integral to the rotor or otherwise coupled to the rotor to fix relative rotational position therebetween (e.g., via a spline, a key, or a threaded bore of the rotor). In some aspects, the impeller can comprise an Archimedes screw. The impeller 40 can further comprise a motor 42 (FIG. 3) that effects rotation of the Archimedes screw. In other aspects, it is contemplated that the impeller can comprise an axial flow pump. In still further aspects, it is contemplated that the impeller can comprise a plurality of blades or paddles that extend radially outwardly from a central body of the impeller. In further exemplary aspects, the impeller can comprise a plurality of vanes that are configured to effect axial flow upon rotation of the plurality of vanes about a rotational axis.

A first anchor 50 can be positioned proximal of the second portion 34 of the body 20. The first anchor 50 can be configured to inhibit movement of the second portion 34 proximally past the aortic valve. In this way, the first anchor 50 can ensure that each opening 32 is positioned within the aorta. In further aspects, the first anchor 50 can be configured to occlude blood flow through the aortic valve outside of the catheter 10 (e.g., proximally, from the aorta into the left ventricle). In exemplary aspects, the first anchor 50 can comprise nitinol. In some aspects, the first anchor can comprise a plate anchor. In some optional aspects, the first anchor 50 can be structurally similar to an atrial closure device, but with the body 20 extending therethrough.

A second anchor 52 can be positioned proximal of the first portion 30 of the body 20. The second anchor 52 can be configured to inhibit movement of the first portion of the body proximally past the septum. In further aspects, the second anchor 52 can be configured to occlude blood flow through the septum. A third anchor 54 can be positioned proximally of the second anchor 52. The third anchor 54 can be configured to inhibit movement of the first portion of the body distally (e.g., into the left ventricle). In further aspects, the third anchor 54 can be configured to occlude blood flow through the septum. In some optional aspects, the second and third anchors 52, 54 can be structurally similar to septal occluders, but with the body 20 extending therethrough. In exemplary aspects, the second and third anchors 52, 54 can comprise nitinol and/or a textile such as dacron or Gore-Tex material. By including an atrial septal anchor pair (second and third anchors 52, 54) that serves a dual purpose to anchor the catheter as well as to occlude the iatrogenic atrial septal defects, thus preventing creation of an iatrogenic right-to-left shunt.

In some aspects, one or more of the first, second, or third anchors can be a deployable anchor that is movable about and between a retracted position and a deployed position. In some aspects, one or more of the first, second, third, or fourth anchors can be non-deployable (passive) anchors. For example, such a non-deployable anchor can comprise a surface feature that inhibits movement across a surface of the patient. Such a surface feature that inhibits movement can comprise one or more radial projections or a relative increase in cross sectional diameter or other dimension of the catheter. In some aspects, only one of the first, second, or third anchors is deployable, and the others are non-deployable. For example, in some optional aspects, the second anchor is a deployable anchor, and the first and third anchors are non-deployable. In other aspects, each of the first, second, and third anchors is a deployable anchor. In other aspects, the second anchor is deployable, and one of the first and third anchors is a deployable anchor, with the other anchor being non-deployable. For example, the second and third anchors can be deployable, and the first anchor can be non-deployable.

It can be particularly advantageous for the second anchor, positioned against the surface of the left aorta, to be deployable in order to ensure that the catheter is not retracted through the IAS.

The body 20 can further comprise an intermediate portion 38 extending between the first portion 30 and the second portion 34. At least a portion of the impeller 40 can be positioned within the intermediate portion 38 of the body 20. In some aspects, an entirety of the impeller 40 can be positioned within the intermediate portion 38 of the body 20.

In some aspects, the catheter 10 can further comprise an anti-traumatic tip 60 distal of the second portion 34 of the body 20. The anti-traumatic tip can be configured to avoid or prevent tissue injury when the tip contacts tissue within the body of the subject. Thus, it is contemplated that anti-traumatic tip 60 does not include sharp edges or other structures that are configured to penetrate or cut tissue. The anti-traumatic tip 60 can comprise, for example, a pigtail structure. In exemplary aspects, the anti-traumatic tip 60 can be flexible. The anti-traumatic tip 60 can have a rounded end. A distal end of the anti-traumatic tip 60 can extend transverse or at an angle from a longitudinal axis of the catheter. In some aspects, the anti-traumatic tip 60 can extend along a curved (e.g., arcuate) path. Optionally, the anti-traumatic tip 60 can curve at least 90 degrees and double backwardly so that the end of the tip is proximal of an intermediate section of the anti-traumatic tip 60.

In some aspects, the catheter 10 can comprise a cable portion 62 that is configured to deliver power to the impeller 40. In some aspects, the cable portion 62 can comprise a braided stainless steel sleeve 64. The braided stainless steel sleeve 64 can provide greater stiffness to the catheter 10 than a portion 65 without the braided stainless steel sleeve. Accordingly, in some aspects, a distal end portion of the catheter can be a flexible catheter to transition from the left atrium (LA) to the aorta (Ao), and a proximal end portion of the catheter can have a greater stiffness. In some aspects, the catheter 10 can comprise gel. For example, the catheter 10 can comprise a gel-based covering (e.g., a coating) that is configured to minimize risk of fracture or damage of wires. The gel-based covering can be provided over a proximal portion (e.g., some or all of the cable portion 62) of the catheter.

In some aspects, the catheter 10 can comprise a first radiopaque marker 66 proximate to (optionally, within) the first portion 30 of the body 20. For example, the first radiopaque marker 66 can be positioned distal of the first portion 30 of the body 20 and can mark where the catheter 10 should be positioned within the mitral valve. Accordingly, with the first radiopaque marker 66 positioned at the mitral valve, the entirety of the first portion 30, and all of the opening(s) 32, can be positioned proximal of the mitral valve and entirely within the left atrium.

The catheter 10 can further comprise a second radiopaque marker 68 proximate to (optionally, within) the second portion 34 of the body 20. For example, the second radiopaque marker 68 can be positioned at or in the first anchor 50. The second radiopaque marker 68 can be used to confirm that the second portion of the body 20, and all of the opening(s) 36, are entirely within the aorta.

In some aspects, the catheter can have a maximum cross sectional outer dimension (e.g., diameter) of at least 18 French, or at least 20 French, or at least 30 French. For example, in some exemplary aspects, the catheter 10 can have a maximum cross sectional outer dimension from 18 French to 26 French, or from 18 French to 30 French. In exemplary aspects, the catheter can have a maximum outer cross sectional dimension of about 30 French. In other exemplary aspects, the catheter can have a maximum outer cross sectional dimension of about 22 French. As should be understood arterially inserted devices cannot have such large cross sectional outer dimensions.

In some aspects, the impeller 40 can be configured to effect a maximum flow rate of at least 5 liters per minute. In further aspects, the impeller 40 can be configured to effect a maximum flow rate of at least 6 liters per minute. In further aspects, the impeller 40 can be configured to effect a maximum flow rate of at least 7 liters per minute. In still further aspects, the impeller 40 can be configured to effect a maximum flow rate of at least 8 liters per minute. In still further aspects, the impeller 40 can be configured to effect a maximum flow rate of at least 10 liters per minute. For example, the impeller can be configured to effect a maximum flow rate from about 6 liters per minute to about 10 liters per minute, or from about 6 liters per minute to about 8 liters per minute, or from about 7 liters per minute to about 10 liters per minute. It is contemplated that the size of the body 20 and the geometry and speed of the impeller 40 can cooperate to determine blood flow rate provided by the catheter 10.

A system 100 can comprise a power supply 102 operably coupled to the impeller. The power supply 102 can comprise, for example, one or more batteries. In further aspects, the system can comprise a wireless charge receiver 104 that is in electrical communication with the power supply 102. The wireless charge receiver 104 can be configured to deliver power to the power supply 102. The wireless charge receiver 104 can further be configured (e.g., sized and shaped) for receipt in a subcutaneous pocket of a patient. The system 100 can further comprise a wireless charge transmitter 106 that is configured to deliver power to the wireless charge receiver 104 (e.g., via induction).

In various aspects, the system can comprise a controller 108 that is configured to control the speed of the impeller. For example, the controller can be in communication with one or more sensors that are configured to detect body motion and/or breathing rate. Based on feedback from said sensors, the controller can then vary the speed of the impeller to provide the desired blow flow rate. For example, high movement speed and/or high breathing rate can indicate a larger required blood flow rate than if the patient were at rest, and the controller can cause the impeller to increase blood flow upon receiving signals from sensors indicative of high movement speed and/or high breathing rate.

In some aspects, one or more of the power supply 102, wireless charge receiver 104, and controller 108 can be integral to the catheter 10. In other aspects, one or more of the power supply 102, wireless charge receiver 104, and controller 108 can be separate from, and coupled to the catheter 10. For example, in some aspects, a back (proximal) end of the catheter can plug into the power supply.

A method of implanting the catheter as described herein is disclosed. In some aspects, the catheter 10 can be positioned via axillary vein access. In other aspects, the catheter can be installed via tunneled intra-jugular access. Accordingly, the catheter 10 can be installed via a vein.

A transseptal puncture can be performed via intracardiac echocardiography (ICE) transseptal puncture or transesophageal echocardiography guided transseptal puncture.

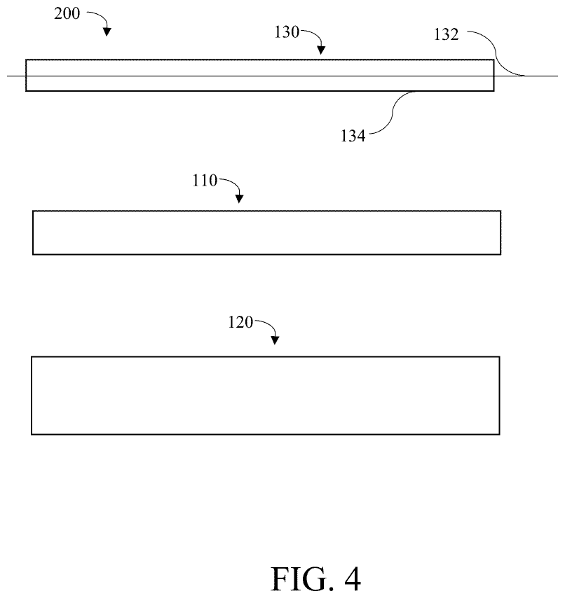

Referring also to FIG. 4, a kit 200 can comprise one or more tools for positioning the catheter 10. A first, small sheath 110 (e.g., a 6 French sheath) can be inserted into the left atrium. The first, small sheath can be exchanged for a second, larger sheath 120. This exchange can be consistent with the standard technique to progressively dilate a pathway, as is known by those skilled in the art. The second, larger sheath can be a peel-away sheath. The second, larger sheath can have a cross sectional dimension (e.g., diameter) of about 24 French. In some aspects, the second, larger sheath 120 can have a cross sectional dimension from about 20 French to about 28 French, or from about 22 French to about 26 French, or about 20 French, or about 21 French, or about 22 French, or about 23 French or about 25 French, or about 25 French, or about 27 French, or about 28 French, or about 29 French, or about 30 French. In various optional aspects, the second, larger sheath can have a length from about 40 cm to about 80 cm (e.g., about 60 cm).

A dilator 130 can be used to advance the second, larger sheath into the aorta. The dilator 130 can be received through the first, small sheath 110. The dilator 130 can be inserted into the aorta via the left ventricle outflow tract (LVOT). The dilator 130 can comprise, for example, a guidewire 132 and a flexible catheter 134. In some aspects, the dilator can be inserted via balloon guidance. In exemplary aspects, the dilator 130 can comprise a slightly smaller catheter than the second, larger sheath (e.g., 23 French flexible catheter). In this way, the dilator can permit positioning of the guidewire in a distal and stable location for later introduction of the second, larger sheath. The guidewire 132 can be sufficiently stiff to permit advancement to the aorta. Accordingly, in some exemplary aspects, the guidewire can have a 0.035 inch diameter. The guidewire 132 can have an anti-traumatic tip (e.g., a J-tip).

The second, larger sheath 120 can be advanced over the dilator until the second, larger sheath extends into the aorta. The dilator 130 can then be removed, thereby leaving the second, larger sheath 120 in position to place the catheter 10.

The catheter 10 can be advanced through the second, larger sheath until the second portion 34 of the elongate body 20 is in the aorta. The second radiopaque marker 68 can be used to confirm the position of the second portion 34. The catheter 10 can further be positioned so that the first portion 22 of the body is within the left ventricle. In some aspects, the first radiopaque marker 66 can be positioned at the mitral valve to confirm that the first portion 22 of the body is within the left ventricle.

The second, larger sheath 120 can be retracted, and the first anchor 50 can be deployed to secure the second portion 34 of the body 20 in the aorta. In some aspects, retraction of the second, larger sheath past the first anchor 50 can cause the first anchor to automatically deploy. In other aspects, the first anchor can be selectively deployed. The first anchor 50 can serve as an atrial occluder.

The second, larger sheath 120 can be retracted to deploy the second anchor the right side of the septum. The catheter 10 can be retracted to pull the second anchor against the septum, thereby occluding blood flow through the septum from the right side. The second, larger sheath can further be retracted to deploy the third anchor on the left side of the septum. The third anchor can occlude blood flow through the septum from the left side.

A transesophageal echocardiogram can be performed to confirm minimal shunting. The second, larger sheath can then be fully removed from the patient.

The catheter 10 can be electrically coupled to the power supply. The impeller can then be turned on.

The wireless charge receiver can be positioned in a subcutaneous pocket of the patient.

Accordingly, in various aspects, a method for positioning the catheter 10 can comprise advancing the catheter through the septum to position the second portion 34 of the body within the aorta and the first portion 30 of the body within the left ventricle. The catheter 10 can be advanced through a sheath. Imaging can be used to determine proper positioning of the catheter 10.

The second, larger sheath can be retracted to deploy the first anchor to inhibit movement of the second portion proximally past the aortic valve. The sheath can further be retracted to deploy the second anchor inhibit movement of the first portion of the elongate body proximally past the septum. The sheath can further be retracted to deploy the third anchor to inhibit movement of the first portion of the elongate body distally.

The disclosed catheter can provide various advantages over conventional methods for supporting patients suffering from serious cardiac disease and in need of mechanical pumping assistance, such as that of a ventricular assist device. For example, smokers are typically unable to survive with a ventricular assist device, whereas smokers can survive implantation and use of the disclosed catheter. Further, unlike the disclosed catheter, the external pump of the ventricular assist device can lead to arterial thrombosis.

Additionally, ventricular assist devices are limited by the size of the artery (which is particularly limiting for female patients), and the speed of the pump is limited, as higher speeds can damage blood cells. Therefore, ventricular assist devices have a maximum flow rate between 2.5 liters per minute and 5 liters per minute. For example, by being implantable within a vein, the disclosed catheter 10 can have a larger bore, thereby providing a substantially higher flow rate, than conventional ventricular assist devices.

Still further, by avoiding thoracic surgery, as required by a ventricular assist device, a patient has a higher likelihood of being able to receive a heart transplant later.

The disclosed catheter can be configured for long-term use. For example, in some aspects, the disclosed catheter can be used for the lifetime of the patient, thereby avoiding the need for a heart transplant.

Users of the disclosed catheter can include NYHA IV patients on home inotropes who are not mechanical circulatory support (MCS) candidates but may hemodynamically benefit from MCS therapy.

### Exemplary Aspects

In view of the described products, systems, and methods and variations thereof, herein below are described certain more particularly described aspects of the invention. These particularly recited aspects should not however be interpreted to have any limiting effect on any different claims containing different or more general teachings described herein, or that the “particular” aspects are somehow limited in some way other than the inherent meanings of the language literally used therein.

Aspect 1: A catheter comprising a portion that is positionable within a heart having a left atrium, an aorta, and a septum, the catheter comprising:
an elongate body having an outer surface and an inner surface defining an interior passage of the elongate body, the elongate body comprising:
   a first portion that is configured to be positioned within a left atrium, the first portion defining at least one opening extending from the outer surface to the inner surface of the elongate body; and
   a second portion that is distal of the first portion and configured to be positioned in the aorta when the first portion is positioned within the left atrium, the second portion defining at least one opening extending from the outer surface to the inner surface of the elongate body;
an impeller that is positioned between the first portion and the second portion, wherein the impeller is configured to effect blood flow through the elongate body from the first portion to the second portion;
a first anchor positioned proximal of the second portion, wherein the first anchor is configured to inhibit movement of the second portion proximally past the aortic valve;
a second anchor positioned proximal of the first portion, wherein the second anchor is configured to inhibit movement of the first portion of the elongate body proximally past the septum; and
a third anchor positioned proximally of the second anchor, wherein the third anchor is configured to inhibit movement of the first portion of the elongate body distally.

Aspect 2: The catheter of aspect 1, wherein the elongate body further comprises an intermediate portion extending between the first portion and the second portion, wherein at least a portion of the impeller is positioned within the intermediate portion.

Aspect 3: The catheter of aspect 1 or aspect 2, wherein the at least one opening defined by the first portion of the elongate body comprise a plurality of openings.

Aspect 4: The catheter of any one of the preceding aspects, wherein the at least one opening defined by the second portion of the elongate body comprise a plurality of openings.

Aspect 5: The catheter of any one of the preceding aspects, wherein the catheter comprises an anti-traumatic tip distal of the second portion.

Aspect 6: The catheter of any one of the preceding aspects, further comprising a cable that is configured to deliver power to the impeller.

Aspect 7: The catheter of aspect 6, wherein the cable comprises a braided stainless steel sleeve.

Aspect 8: The catheter of any one of the preceding aspects, further comprising a first radiopaque marker proximate to the first portion of the elongate body.

Aspect 9: The catheter of any one of the preceding aspects, further comprising a second radiopaque marker proximate to the second portion of the elongate body.

Aspect 10: The catheter of any one of the preceding aspects, wherein the catheter has a maximum cross sectional outer dimension from 18 French to 30 French.

Aspect 11: The catheter of any one of the preceding aspects, wherein the catheter has a maximum cross sectional outer dimension of from 22 French to 30 French.

Aspect 12: The catheter of any one of the preceding aspects, wherein the impeller is configured to effect a maximum flow rate of at least 5 liters per minute.

Aspect 13: The catheter of any one of the preceding aspects, wherein the impeller is configured to effect a maximum flow rate of at least 6 liters per minute.

Aspect 14: The catheter of any one of the preceding aspects, wherein the impeller is configured to effect a maximum flow rate of at least 8 liters per minute.

Aspect 15: A system comprising:
a catheter as in any one of the preceding aspects; and
a power supply operably coupled to the impeller.

Aspect 16: The system of aspect 15, further comprising a wireless charge receiver that is in electrical communication with the power supply, wherein the wireless charge receiver is configured to deliver power to the power supply.

Aspect 17: The system of aspect 16, further comprising a wireless charge transmitter that is configured to deliver power to the wireless charge receiver.

Aspect 18: The system of any one of aspects 15-17, further comprising a controller that is configured to control the speed of the impeller.

Aspect 19: A method of positioning the catheter as in any one of aspects 1-14, the method comprising:
advancing the catheter through the septum to position the second portion of the elongate body within the aorta and the first portion of the elongate body within the left ventricle.

Aspect 20: The method of aspect 19, wherein advancing the catheter comprises advancing the catheter through a sheath.

Aspect 21: The method of aspect 20, further comprising using imaging to determine proper positioning of the catheter.

Aspect 22: The method of aspect 21, further comprising:
retracting the sheath to deploy the first anchor to inhibit movement of the second portion proximally past the aortic valve;
retracting the sheath to deploy the second anchor inhibit movement of the first portion of the elongate body proximally past the septum; and
retracting the sheath to deploy the third anchor to inhibit movement of the first portion of the elongate body distally.

Although the foregoing invention has been described in some detail by way of illustration and example for purposes of clarity of understanding, certain changes and modifications may be practiced within the scope of the appended claims.

What is claimed is:

1. A catheter having a portion that is positionable within a heart having a left atrium, an aorta, and a septum, the catheter comprising:
an elongate body having an outer surface and an inner surface defining an interior passage of the elongate body, the elongate body comprising:
a first portion that is configured to be positioned within a left atrium, the first portion defining at least one opening extending from the outer surface to the inner surface of the elongate body; and
a second portion that is distal of the first portion and configured to be positioned in the aorta when the first portion is positioned within the left atrium, the second portion defining at least one opening extending from the outer surface to the inner surface of the elongate body;
an impeller that is positioned between the first portion and the second portion, wherein the impeller is configured to effect blood flow through the elongate body from the first portion to the second portion;
a first anchor positioned proximal of the second portion, wherein the first anchor is configured to inhibit movement of the second portion proximally past the aortic valve;
a second anchor positioned proximal of the first portion, wherein the second anchor is configured to inhibit movement of the first portion of the elongate body proximally past the septum; and
a third anchor positioned proximally of the second anchor, wherein the third anchor is configured to inhibit movement of the first portion of the elongate body distally.

2. The catheter of claim 1, wherein the elongate body further comprises an intermediate portion extending between the first portion and the second portion, wherein at least a portion of the impeller is positioned within the intermediate portion.

3. The catheter of claim 1, wherein the at least one opening defined by the first portion of the elongate body comprises a plurality of openings.

4. The catheter of claim 1, wherein the at least one opening defined by the second portion of the elongate body comprises a plurality of openings.

5. The catheter of claim 1, wherein the catheter comprises an anti-traumatic tip distal of the second portion.

6. The catheter of claim 1, further comprising a cable that is configured to deliver power to the impeller.

7. The catheter of claim 6, wherein the cable comprises a braided stainless steel sleeve.

8. The catheter of claim 1, further comprising a first radiopaque marker proximate to the first portion of the elongate body.

9. The catheter of claim 1, further comprising a second radiopaque marker proximate to the second portion of the elongate body.

10. The catheter of claim 1, wherein the catheter has a maximum cross sectional outer dimension from 18 French to 30 French.

11. The catheter of claim 1, wherein the catheter has a maximum cross sectional outer dimension from 22 French to 30 French.

12. The catheter of claim 1, wherein the impeller is configured to effect a maximum flow rate of at least 5 liters per minute.

13. A system comprising:
a catheter having a portion that is positionable within a heart having a left atrium, an aorta, and a septum, the catheter comprising:
an elongate body having an outer surface and an inner surface defining an interior passage of the elongate body, the elongate body comprising:
a first portion that is configured to be positioned within a left atrium, the first portion defining at least one opening extending from the outer surface to the inner surface of the elongate body; and
a second portion that is distal of the first portion and configured to be positioned in the aorta when the first portion is positioned within the left atrium, the second portion defining at least one opening extending from the outer surface to the inner surface of the elongate body;
an impeller that is positioned between the first portion and the second portion, wherein the impeller is configured to effect blood flow through the elongate body from the first portion to the second portion;
a first anchor positioned proximal of the second portion, wherein the first anchor is configured to inhibit movement of the second portion proximally past the aortic valve;
a second anchor positioned proximal of the first portion, wherein the second anchor is configured to inhibit movement of the first portion of the elongate body proximally past the septum; and
a third anchor positioned proximally of the second anchor, wherein the third anchor is configured to inhibit movement of the first portion of the elongate body distally; and
a power supply operably coupled to the impeller.

14. The system of claim 13, further comprising a wireless charge receiver that is in electrical communication with the power supply, wherein the wireless charge receiver is configured to deliver power to the power supply.

15. The system of claim 14, further comprising a wireless charge transmitter that is configured to deliver power to the wireless charge receiver.

16. The system of claim 13, further comprising a controller that is configured to control the speed of the impeller.

17. A method of positioning a catheter, the catheter having a portion that is positionable within a heart having a left atrium, an aorta, and a septum, the catheter comprising:
an elongate body having an outer surface and an inner surface defining an interior passage of the elongate body, the elongate body comprising:
a first portion that is configured to be positioned within a left atrium, the first portion defining at least one opening extending from the outer surface to the inner surface of the elongate body; and
a second portion that is distal of the first portion and configured to be positioned in the aorta when the first portion is positioned within the left atrium, the second portion defining at least one opening extending from the outer surface to the inner surface of the elongate body;
an impeller that is positioned between the first portion and the second portion, wherein the impeller is configured to effect blood flow through the elongate body from the first portion to the second portion;
a first anchor positioned proximal of the second portion, wherein the first anchor is configured to inhibit movement of the second portion proximally past the aortic valve;
a second anchor positioned proximal of the first portion, wherein the second anchor is configured to inhibit movement of the first portion of the elongate body proximally past the septum; and
a third anchor positioned proximally of the second anchor, wherein the third anchor is configured to inhibit movement of the first portion of the elongate body distally, the method comprising:
advancing the catheter through the septum to position the second portion of the elongate body within the aorta and the first portion of the elongate body within the left ventricle.

18. The method of claim 17, wherein advancing the catheter comprises advancing the catheter through a sheath.

19. The method of claim 18, further comprising using imaging to determine proper positioning of the catheter.

20. The method of claim 19, further comprising:
retracting the sheath to deploy the first anchor to inhibit movement of the second portion proximally past the aortic valve;
retracting the sheath to deploy the second anchor inhibit movement of the first portion of the elongate body proximally past the septum; and
retracting the sheath to deploy the third anchor to inhibit movement of the first portion of the elongate body distally.
